(19) United States
(12) Patent Application Publication
(10) Pub. No.: US 2010/0256494 A1
(43) Pub. Date: Oct. 7, 2010

(54) ULTRASONIC IMAGING SYSTEM

(76) Inventor: Takashi Azuma, Sagamihara (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/742,468

(22) PCT Filed: Sep. 24, 2008
(86) PCT No.: PCT/JP2008/067160

§ 371 Date: May 12, 2010

(30) Foreign Application Priority Data
Nov. 16, 2007 (JP) 2007297891

(51) Int. Cl. A61B 8/14

(52) U.S. Cl. 600/443

(57) ABSTRACT

An ultrasonic imaging system is provided that, when a deviation occurs between a predicted tissue moving direction and a displacement searching direction, can decrease an error caused by the deviation and thereby improve the accuracy of an elasticity image. An elastographic image in which a deviation in a displacement direction is corrected is created based on an RF displacement relating to an ultrasonic wave propagation direction that is calculated based on a cross-correlation between RF signals, and an ultrasonic wave propagation direction component map of applied pressure that uses a correction angle map determined based on a vector displacement map obtained by performing block matching between two-dimensional video images. According to this method, an image of an elasticity ratio can be acquired without a decrease in accuracy even if a tissue displacement vector deviates from the orientation of a normal line vector of a wave transmitting surface of an ultrasonic probe.

### TECHNICAL FIELD

The present invention relates to ultrasound imaging technology that images an elasticity image that shows characteristics such as strain or elasticity of biological tissue of an object.

### BACKGROUND ART

Known ultrasonic diagnostic apparatuses include an apparatus that images an elasticity image that shows characteristics such as strain or elasticity of biological tissue of an object (for example, see Patent Document 1).

Normally, a point spread function in ultrasonic imaging is short in the propagation direction of ultrasonic waves and is wide in a direction perpendicular to the propagation direction (hereunder, referred to as “lateral direction”). Hence, for a local displacement measurement, a measurement that relates only to the propagation direction is performed. In practice, there are situations in which a direction in which biological tissue is actually displaced (hereunder, referred to as “tissue moving direction”) when pressure is applied to an object and an elasticity calculation direction (hereunder, referred to as “displacement searching direction”) that measures a displacement of biological tissue are not necessarily parallel. To cope with this kind of situation, a method is available that matches the displacement searching direction with the tissue moving direction (for example, see Patent Document 2).

### Patent Document 1: JP Patent Publication (Kokai) No. 2004-57653A

### Patent Document 2: International Patent Publication No. 2006/073088

### DISCLOSURE OF THE INVENTION

### Problems to be Solved by the Invention

According to the prior art as described above, there is the unsolved problem that when the moving direction of biological tissue involves a more complicated motion, a deviation arises between the predicted tissue moving direction and the displacement searching direction.

An object of the present invention is to provide an ultrasonic imaging system that, when a deviation occurs between a predicted tissue moving direction and a displacement searching direction, can decrease an error caused by the deviation and thereby improve the accuracy of an elasticity image.

### Means for Solving the Problems

An ultrasonic imaging system of the present invention includes: an ultrasonic probe that transmits an ultrasonic wave at an object and receives a reflection echo; an RF signal processor that acquires a first RF raster signal corresponding to an ultrasonic wave that is transmitted before a deformation of interest of an object, and a second RF raster signal corresponding to an ultrasonic wave that is transmitted after the deformation of interest; a processor of displacement estimated with RF signals that acquires a displacement in a raster direction of each portion of an object based on the first RF raster signal and second RF raster signal; a two dimensional displacement processor that estimates a two-dimensional displacement vector that shows a displacement of each portion of an object before and after the deformation of interest; an applied pressure estimated value correction part that corrects an applied pressure estimated value produced by the deformation of interest in correspondence with a direction of the estimated two-dimensional displacement vector and an ultrasonic wave irradiation direction; an elasticity estimation processor that estimates an elasticity of each portion of the object based on the corrected applied pressure estimated value and the two-dimensional displacement vector; and a display that displays elasticity information that is estimated by the elasticity estimation processor.

### ADVANTAGE OF THE INVENTION

According to the present invention, an ultrasonic imaging system can be provided that, when a deviation occurs between a predicted tissue moving direction and a displacement searching direction, can decrease an error caused by the deviation and thereby improve the accuracy of an elasticity image.

### BRIEF DESCRIPTION OF THE DRAWINGS

FIG. 1 is a schematic explanatory view of ultrasonic imaging.

FIG. 2 is a block diagram illustrating a configuration example of an ultrasonic diagnostic apparatus.

FIG. 3 is a flowchart of ultrasonic imaging processing.

FIG. 4 is a view that describes the relation between a displacement searching direction and a tissue moving direction.

FIG. 5 is a view illustrating an example of one set of RF raster signals.

FIG. 6 is an explanatory view of processing that carries out a displacement estimation by shifting depths that set a cross correlation window.

FIG. 7 is an explanatory view of a block matching method.

FIG. 8 is an explanatory view of processing that shifts positions of a cross correlation block and a searching area to determine a displacement vector.

FIG. 9 is a block diagram that illustrates a configuration example of an ultrasonic diagnostic apparatus.

FIG. 10 is a block diagram that illustrates a configuration example of an ultrasonic diagnostic apparatus.

FIG. 11 is an explanatory view of bidirectional ultrasonic transmission.

FIG. 12 is a flowchart of ultrasonic imaging processing.

FIG. 13 is a block diagram that illustrates a configuration example of an ultrasonic diagnostic apparatus.

FIG. 14 is a flowchart of ultrasonic imaging processing.

FIG. 15 is an explanatory view of another example relating to ultrasonic transmission directions.

### DESCRIPTION OF SYMBOLS

100 elasticity image processor
101 object
102 probe
103 ultrasonic transmit/receive part
104 RF signal processor
105 video signal processor
106 tomography DSC
108 processor of displacement estimated with RF signals
110 two dimensional displacement processor
111 correction angle estimation processor
112 correction part of estimated applied pressure value
113 strain estimation processor
114 elasticity estimation processor
115 color DSC
116 controller
117 user interface
118 image synthesizer
119 display
120 correction value processor for applied pressure
121 strain correction processor
301 tissue moving direction
302 displacement searching direction
500 aperture
501 raster
502 cross correlation window
503 searching area
504 cross correlation block
505 searching area
506 displacement
507 displacement vector

### BEST MODE FOR CARRYING OUT THE INVENTION

Examples of embodiments of the present invention are described below.

### Embodiment 1

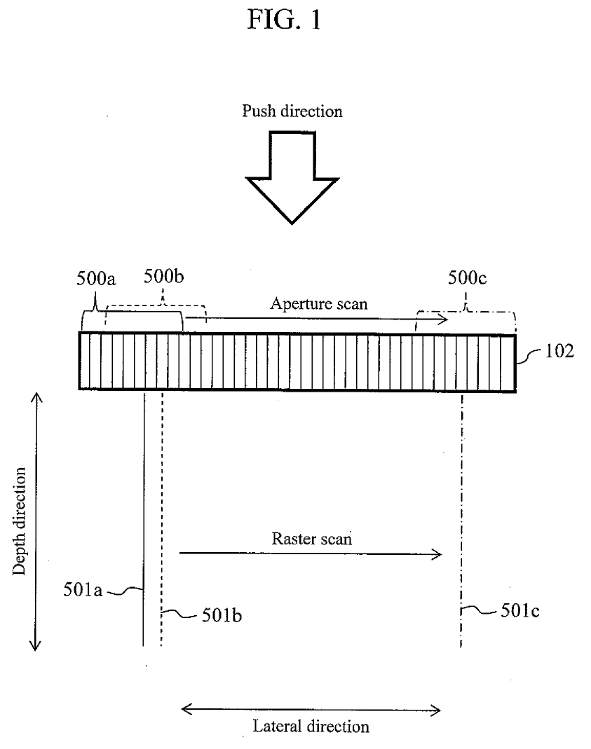

First, an outline of ultrasonic imaging is described using FIG. 1. Transmitting and receiving is performed using an aperture 500a inside an ultrasonic probe 102 to thereby acquire echo data on a raster 501a. The raster direction is called “depth direction” hereafter. When acquisition of echo data on the raster 501a ends, the transmitting and receiving operation shifts to an aperture 500b to acquire echo data on a raster 501b, and aperture shifting and shifting of the acquisition raster is repeated until an aperture 500c that corresponds to a raster 501c, to thereby acquire echo data for a single frame. The direction in which the rasters are aligned is referred as the “lateral direction”. The frame rate of the ultrasonic imaging is determined by:

(time required to acquire echo data on a single raster)×(number of rasters)

The time required to obtain echo data on a single raster is given by (distance both ways/sound velocity). Since the sound velocity with respect to a living organism is approximately constant, the time required to acquire data of a single raster is determined upon deciding the field of view. Therefore, when imaging the movement of a living organism at a frame rate that can be tracked, the number of rasters is limited and is normally approximately 100 to 200. Consequently, although a sampling interval can be made minute without leading to a decrease in the frame rate in the depth direction, a sampling interval can not be made minute in the lateral direction. When examining a deformation of an object, although measurement can be carried out at a high accuracy in the depth direction, the accuracy becomes poor in the lateral direction. Thus, when forming an elasticity image, imaging is normally performed so as to match the direction of pressure and the depth direction.

A feature of the present invention is that an angle of a displacement is estimated based on a high-accuracy one dimensional displacement estimation and a two dimensional displacement estimation. Although a high accuracy is achieved by the high-accuracy one dimensional displacement estimation, only a displacement in an ultrasonic wave propagation direction can be estimated. On the other hand, when the two dimensional displacement estimation is used, a displacement can be obtained as a vector. Two parameters are necessary to estimate elasticity, namely, the displacement and the pressure amount. According to the present invention, when a high-accuracy displacement estimation direction which is parallel to an ultrasonic wave propagation direction and a pressure vector that causes a displacement are not parallel, a displacement estimation direction component is extracted from the pressure vector, and the elasticity is estimated using the displacement estimation direction component of the pressure vector and a high-accuracy displacement estimation value.

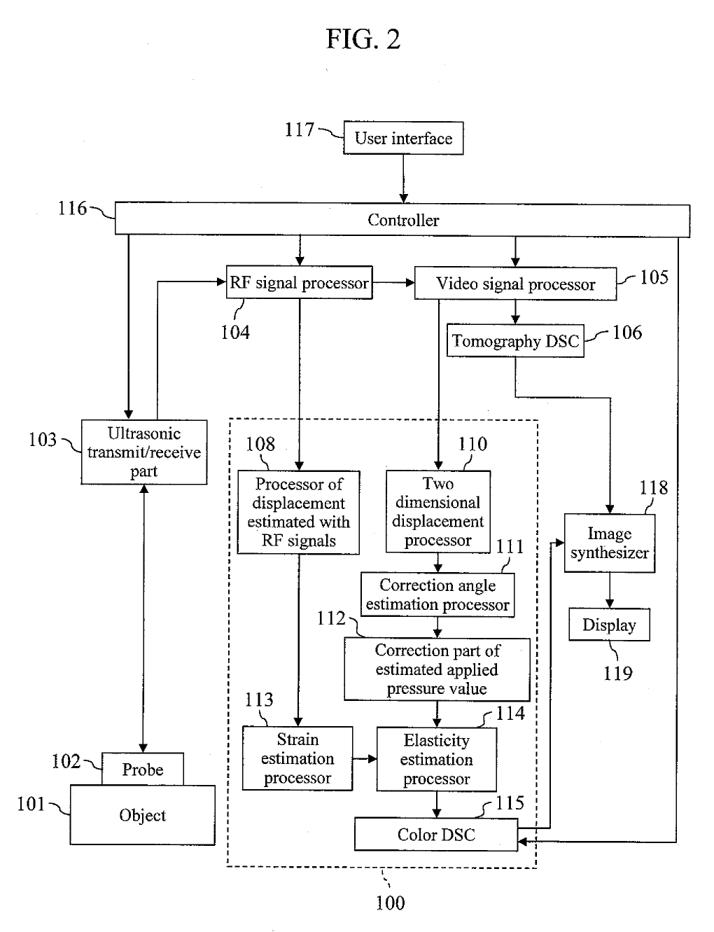

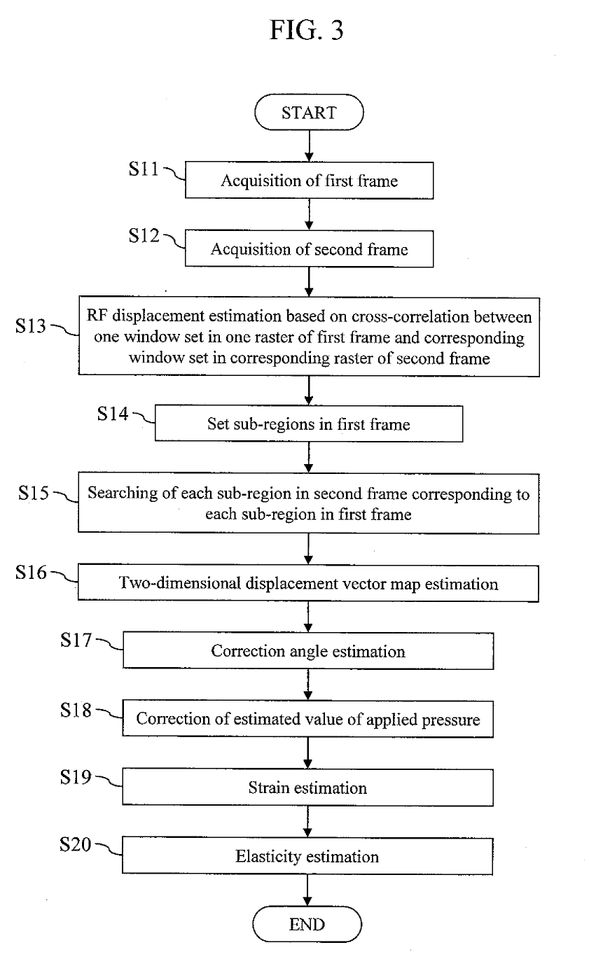

Hereunder, embodiments of an ultrasonic diagnostic apparatus and an ultrasonic imaging method to which the present invention is applied are described referring to the drawings. FIG. 2 is a block diagram of an ultrasonic diagnostic apparatus according to the present embodiment. FIG. 3 is a flowchart of processing performed in the apparatus illustrated in FIG. 2.

As shown in FIG. 2, an ultrasonic diagnostic apparatus includes an ultrasonic probe (hereunder, referred to as “probe”) 102 that transmits and receives ultrasonic waves to and from an object 101, an ultrasonic transmit/receive part 103 that supplies a transmission driving signal to the probe 102 and also processes a received signal that is output from the probe 102, an RF signal processor 104 that processes an output signal of the ultrasonic transmit/receive part 103, a video signal processor 105 that converts an RF signal into a video signal, a tomography digital scan converter (hereunder, referred to as “tomography DSC”) 106 that forms a tomogram from a video signal, an elasticity image processor 100 that creates an elasticity image based on a displacement of a biological tissue that is measured based on an output signal of the ultrasonic transmit/receive part 103, and a display 119 as display means that displays an elasticity image. The elasticity image processor 100 includes a processor of displacement estimated with RF signals 108, a two dimensional displacement processor 110, a correction angle estimation processor 111, a correction part of estimated applied pressure value 112, a strain estimation processor 113, an elasticity estimation processor 114, and a color digital scan converter (hereunder, referred to as “color DSC”) 115. The ultrasonic diagnostic apparatus is also provided with a controller 116 that outputs a control command to the ultrasonic transmit/receive part 103 and the elasticity image processor 100 and the like.

Next, the processing flow is described using FIG. 3. First, image data of a first frame is acquired (S11). Next, image data of a second frame is acquired (S12). It is assumed that, a tissue displacement occurs at a portion of the object due to an applied pressure in a period between the first frame and second frame. Although a pressure is typically applied by the probe 102 pressing the object, for example, the pressure may also be caused by arterial pulsations. A displacement at each depth is estimated by a cross-correlation operation with respect to data for rasters and depths that correspond to RF data obtained in the preceding two steps (S13). In this case, a cross-correlation operation for functions f1(x) and f2(x) is expressed by ∫f1(ν)f2*(ν−x)dν. Next, video image data of the first frame (data for which envelope detection of RF data has been performed, and which has undergone log compression and depth direction resampling processing) is divided into two-dimensional sub-regions (S14). Searching areas that correspond to the sub-regions are set in the image data of the second frame, and a search is performed for a sampling position in the second frame at which the sum of absolute difference is smallest (S15). A movement amount of a sub-region obtained as a search result is estimated as a two-dimensional displacement vector (S16). To estimate an ultrasonic wave propagation direction component of the displacement based on the two-dimensional displacement vector obtained in step 16, an angle α formed between the displacement direction and the ultrasonic wave propagation direction is determined.

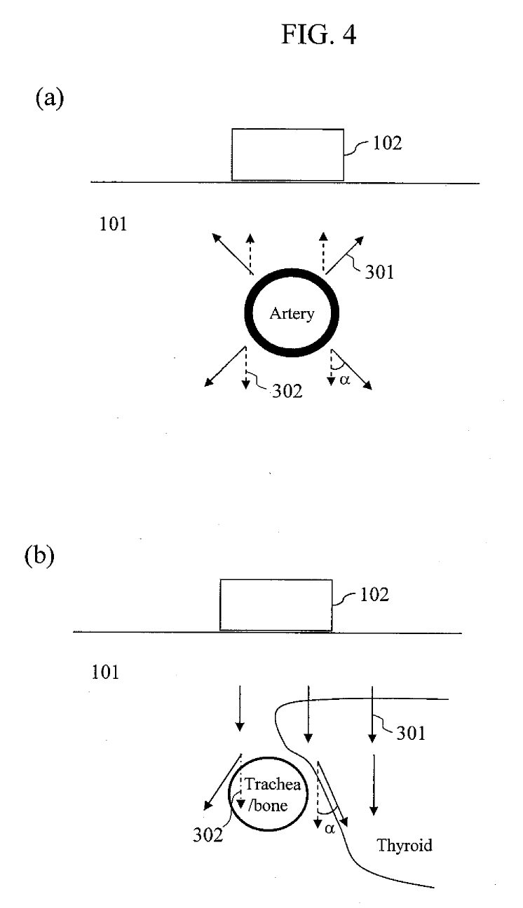

The angle α is described next using FIG. 4. As shown in FIG. 4(a), there are cases in which a tissue displacement occurs due to arterial pulsations. Further, as shown in FIG. 4(b), moving directions 301 of tissue may not be uniform because of the presence of regions with different elasticity such as trachea or bone in the vicinity of a target area. In such case, even if the amount of pressure applied by the ultrasonic probe 102 is constant, a pressure component in the ultrasonic wave propagation direction (displacement searching direction) 302 is not constant. In the elasticity measurement described below, it is assumed that a pressure amount is constant, and the elasticity is determined based on the pressure amount and a strain amount. Hence, when the pressure amount is not constant there is the possibility that the accuracy of estimating the elasticity will decrease. According to the present invention, in addition to the conventional displacement measurement (relating to the ultrasonic wave propagation direction) based on RF signals, a displacement vector map is determined based on a two-dimensional video image, a displacement direction is estimated, and an angle formed between a pressure direction and a moving direction is obtained as a correction angle α (S17). An estimated applied pressure value is corrected based on the correction angle α (S18).

After correction of the estimated applied pressure value, the strain (S19) and the elasticity (S20) are estimated in a similar manner to estimating the elasticity according to the conventional ultrasound elastography. In this case, if a displacement is taken as ΔL, since a strain S is a spatial derivative of the displacement, the strain S is obtained as S=ΔL/Δx. If it is assumed that a modulus of elasticity E equalizes a stress ΔP, the modulus of elasticity E can be calculated as E=ΔP/S.

In this connection, there are many cases in which it is difficult to determine the actual value of ΔP. However, if a spatial change in ΔP in an image is small in comparison to a spatial change in S or E, the distribution of E in the image can be determined in a state in which true E is multiplied by a constant coefficient. Although it is generally difficult to determine the coefficient ε, when performing imaging with respect to a modulus of elasticity, the most important point is that portions which have a different modulus of elasticity in an image are presented in a form in which the shape thereof can be visually identified. Therefore, even if a value of a modulus of elasticity can not be presented, the method is sufficiently useful as a diagnostic imaging method. With respect also to angle correction, which is a feature of the present invention, the purpose is not to determine the coefficient ε, but rather to correct changes within the image of ΔP.

The ultrasonic diagnostic apparatus of the present embodiment is described in further detail below. The constituent elements of the ultrasonic diagnostic apparatus are broadly divided into an ultrasonic transmit/receive system, a tomographic imaging system, an elasticity-image imaging system, a display system, and a control system. The ultrasonic transmit/receive system includes the probe 102 and the ultrasonic transmit/receive part 103. The probe 102 has an ultrasonic wave transmitting and receiving surface that transmits and receives ultrasonic waves to and from the object 101 by performing mechanical or electronic beam scanning. A plurality of transducers is provided in an aligned manner on the ultrasonic wave transmitting and receiving surface. Each transducer converts between electrical signals and ultrasonic waves.

The ultrasonic transmit/receive part 103 includes transmitting means that supplies a transmission driving signal (pulse) to the probe 102 via transmitting/receiving means, and receiving means that processes a received signal that is output from the probe 102 via the transmitting/receiving means.

The transmitting means of the ultrasonic transmit/receive part 103 has a circuit that, at set intervals, transmits a transmission pulse as a driving signal that drives a transducer of the probe 102 to generate an ultrasonic wave, and a circuit that sets a depth of a convergent point of an ultrasonic transmission beam emitted from the probe 102. In this case, the transmitting means of the present embodiment selects a transducer group to supply a pulse via the transmitting/receiving means, and also controls a generation timing of a transmission pulse so that an ultrasound beam transmitted from the probe 102 is scanned in the tissue moving direction. More specifically, the transmitting means is configured to control a scanning direction of an ultrasound beam by controlling a delay time of the pulse signal.

The receiving means of the ultrasonic transmit/receive part 103 includes a circuit that generates an RF signal by amplifying with a predetermined gain a signal that is output from the probe 102 via the transmitting/receiving means, that is, that generates a reception echo signal, and a circuit that subjects phases of RF signals to phasing addition to generate RF signal data in time series. The receiving means applies a predetermined delay time to a received echo signal acquired by means of an ultrasound beam transmitted from the probe 102 via the transmitting/receiving means, and aligns the phases to perform phasing addition.

The tomographic imaging system includes the RF signal processor 104, the video signal processor 105, and the tomography DSC 106. The RF signal processor subjects an RF signal output from the ultrasonic transmit/receive part 103 to low-pass filter processing and frequency shift processing to create complex RF data. Conversion to an absolute value is performed using a root sum of squares based on the complex RF data, the data amount is compressed by resampling the data on a time axis, and log compression processing is further performed at the video signal processor 105 to construct grayscale tomographic data (for example, monochrome tomographic data) relating to the object 101. Further, if needed, gain correction and contour emphasis and the like may be performed during this processing. The tomography DSC 106 reads out tomographic data relating to the object 101 that is stored in a frame memory in frame units, and outputs the read tomographic data with television synchronizing.

The elasticity-image imaging system is provided as an input part of data from both an RF raster memory that is arranged to be branched from the RF signal processor 104 of the tomographic imaging system of the ultrasonic transmit/receive part 103, and a frame memory that is arranged to be branched from the video signal processor 105 of the same tomographic imaging system. In FIG. 3, the RF raster memory is contained in the processor of displacement estimated with RF signals 108, and the frame memory is contained in the two dimensional displacement processor 110.

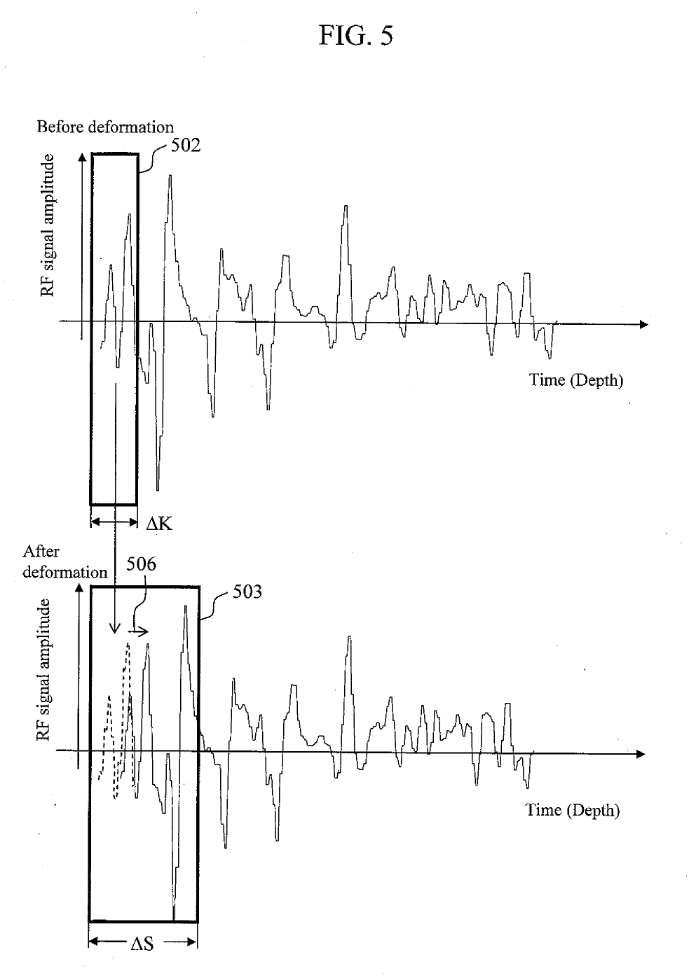

The processor of displacement estimated with RF signals 108 measures a displacement relating to an ultrasonic wave propagation direction in biological tissue of the object 101 based on RF signal data that is output from the ultrasonic transmit/receive part 103. The processor of displacement estimated with RF signals 108 includes an RF signal selection part, a calculation part, and a filter part. The RF signal selection part selects, by means of a selecting part, a set of RF raster signals in frames that are adjacent on two time axes from the RF raster memory that stores RF signal data in time series that is output from the ultrasonic transmit/receive part 103. An example of a set of RF raster signals is shown in FIG. 5. Next, the RF signal selection part sets a cross correlation window 502 that limits a depth in the RF raster signal of the first frame, and sets a searching area 503 that limits a depth of an RF raster signal in the second frame.

The above processing is described hereafter using a mathematical formula. Hereunder, RF data from sampling points k₁ to k₂ in the depth direction for i frame and j raster is expressed as a wave (k₁ to k₂, j, i). For example, regarding an RF displacement disp (K, J, 1) with respect to J raster and a depth (ultrasonic wave propagation direction) K between a first frame and a second frame, a cross-correlation function between the two vectors (RF data) consisting of wave (K−ΔK/2 to K+ΔK/2, J, 1) and wave (K−ΔS/2 to K+ΔS/2, J, 2) is obtained, and a change at a position that takes the largest value thereof is treated as a displacement 506.

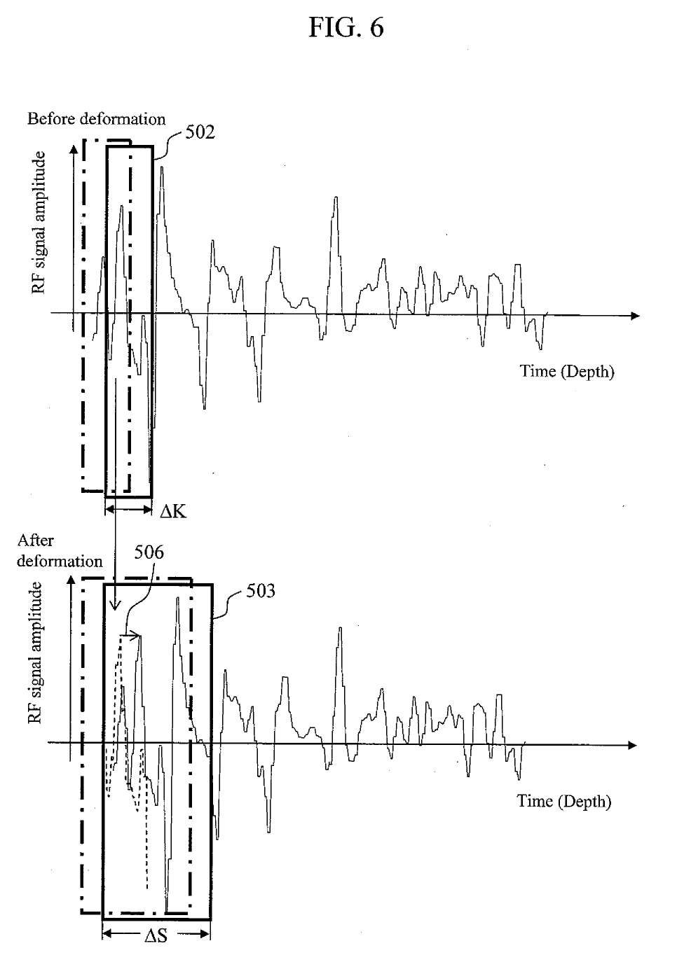

Processing to determine a displacement will now be described referring to the drawings. The processing searches for a waveform whose shape is nearest to that of a cut-out signal within the cross correlation window 502 from an RF signal of the first frame, among signals cut out with the searching area 503 from RF signals of the second frame. A deviation from the position of the cross correlation window 502 to the position of the most similar waveform extracted from the searching area 503 is the displacement. In this case, ΔK denotes the width of the cross correlation window 502 and ΔS denotes the width of the searching area 503. The width ΔS is larger than the width ΔK by the maximum movement amount between the frames in the calculated range. Because the signal-noise ratio deteriorates even though the spatial resolution improves in accordance with a decrease in ΔK, an appropriate value for ΔK is selected depending on the signal. Regarding ΔS, if ΔS is too large the calculation cost increases, while if ΔS is too small the searching area becomes smaller than the displacement maximum value and there is the possibility that an appropriate displacement estimation can not be made. Upon completing the estimation of a displacement at a certain depth, as shown in FIG. 6, the depth that sets the cross correlation window 502 is shifted, the corresponding searching area 503 is set, and displacement estimation is performed again. When displacement estimations relating to all depths are completed by shifting the position of the cross correlation window and the searching area as far as the deepest position in this way, the operation moves to the neighboring raster. By performing this operation for all rasters, the displacement for one frame is estimated.

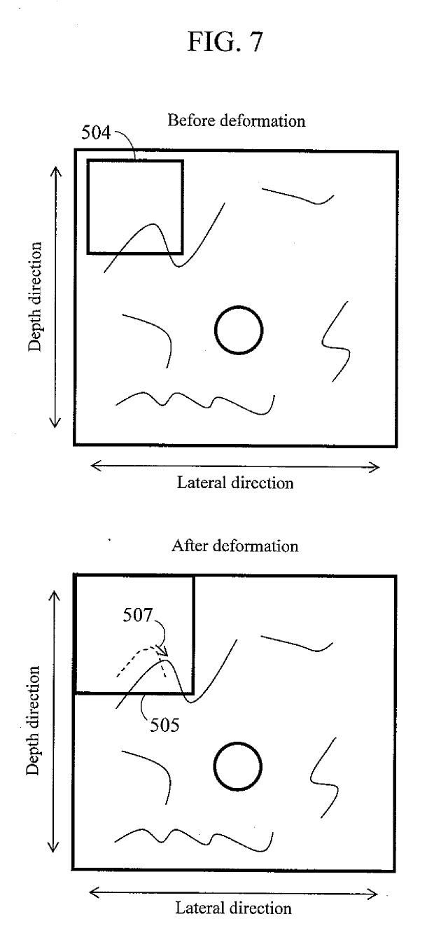

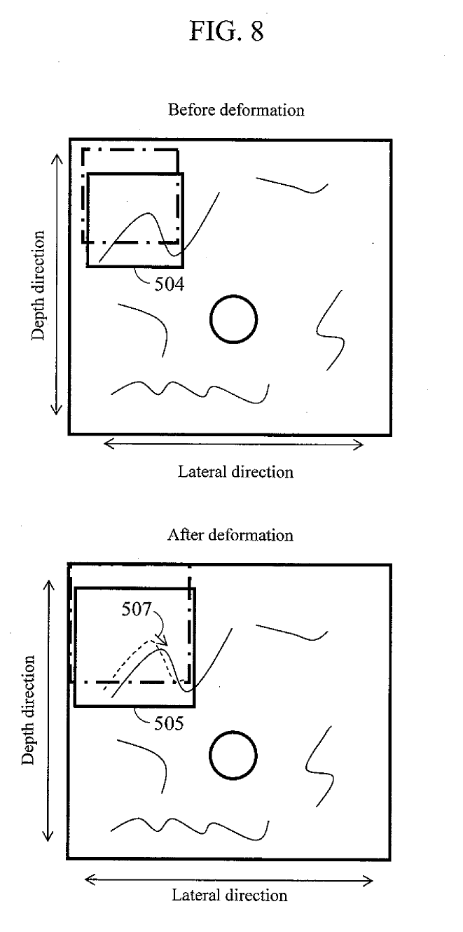

The calculation of the two dimensional displacement processor 110 determines a displacement or displacement vector (hereunder, generically referred to as “displacement” of the biological tissue in the displacement searching direction corresponding to each pixel of the tomogram, for example, by applying a block matching method as the correlation processing. Herein, the term “displacement vector” refers to a two-dimensional displacement distribution relating to the direction and size of the displacement. The term “block matching method” refers to a method that, as shown in FIG. 7, divides an image into cross correlation blocks 504 containing, for example, N×N pixels, searches for an area that most resembles the cross correlation block 504 of interest in the searching area 505 of an adjacent frame, and determines a displacement vector 507 of the cross correlation block 504 as the movement of the center point of the cross correlation block. Methods for searching for an area that resembles a cross correlation block of interest include a method that searches for an area in which the sum total of absolute values of difference values between pixels is smallest, and a method that determines the movement of a block by means of a two-dimensional cross-correlation function. When the displacement vector 507 is determined for a certain cross correlation block, as shown in FIG. 8, the positions of the cross correlation block 504 and the corresponding searching area 505 are shifted, and a displacement vector is determined by means of a similar search. A displacement vector map is obtained by performing this operation for all areas inside the frame. On the displacement vector map are recorded a displacement vector that takes pixel coordinates (x, y) of the tomogram as variables and an angle α (x, y) formed in the ultrasonic wave irradiation direction.

The strain estimation processor 113 estimates strain data (S=ΔL/ΔX) of the biological tissue by spatially differentiating the movement amount of the biological tissue, for example, a displacement ΔL, output from the processor of displacement estimated with RF signals 108. Further, the elasticity estimation processor 114 estimates data relating to the elasticity of the biological tissue by dividing the change in the pressure by the change in the displacement. The elasticity estimation processor 114 corrects an ultrasonic wave propagation direction component of a pressure generated by irregularities in the moving direction based on the result of the correction angle estimation processor 111 with respect to a pressure Δp applied to the ultrasonic wave transmitting and receiving surface of the probe 102, to determine, for example, (ΔP×cos α)/S as elasticity data based on the pressure Δp and displacement ΔL. More specifically, elasticity data at given pixel coordinates (x, y) of the biological tissue is determined as follows.

(ΔP(x, y)×cos α(x, y))/S(x, y)

Thus, the elasticity estimation processor 114 acquires two-dimensional elasticity image data by determining the respective elasticity data that corresponds to each point of the tomogram. Herein, strain data and elasticity data are generically referred to as elasticity data as appropriate. In this connection, it is assumed that ΔP is constant or may be approximated as a function of the distance from the probe 102.

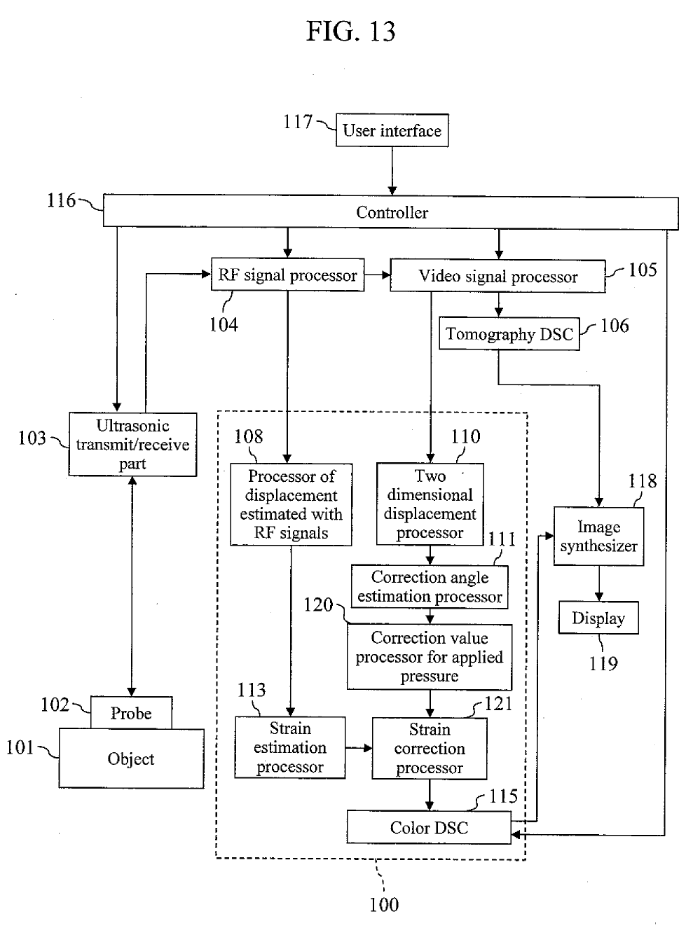

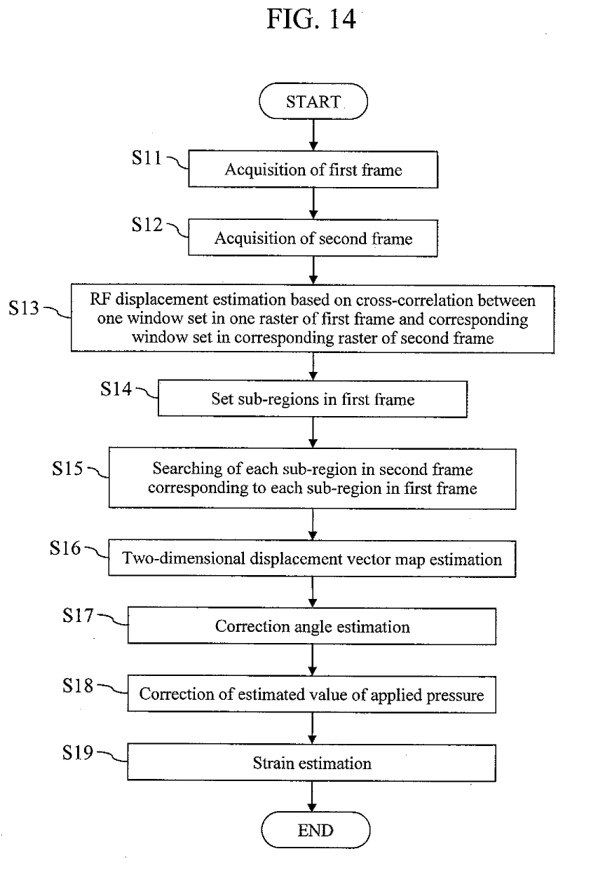

The foregoing description relates to a case in which the elasticity illustrated in FIG. 2 is corrected and displayed. Next, a case in which a correction value that allows an applied pressure amount to be considered as uniform is applied to the strain is described using FIGS. 13 and 14. FIG. 13 is a block diagram. FIG. 14 is a flowchart. A strain when the pressure is uniform is taken as S′ with respect to a strain S determined by actual measurement. If a cause of the applied pressure amount losing its uniformity at this time can be explained as an effect of the pressure vector deviating at the angle α, then it can be expressed that S(x,y)=S′(x,y)×cos α(x,y). That is, if the actual measurement value S and the angle correction amount α are known, the correction S′(x,y)=S(x,y)/cos α(x,y) can be made. Naturally, from the view point of exact physics, the pressure and strain are in a tensor relation, and this kind of correction is difficult. However, there is practical significance in correcting a deviation with from a case in which an ideal uniform pressure can be realized, with respect to a spatial distribution image of strain that is obtained in a state in which spatial variations of the strain and spatial variations of the pressure are mixed.

The color DSC 115 constructs a color elasticity image relating to biological tissue of the object 101 based on strain data output from the strain estimation processor 113 or elasticity data output from the elasticity estimation processor 114. The color scan converter of the color DSC 115 is a color tone converting part that executes color tone converting processing with respect to elasticity data output from the elasticity estimation processor 114 on the basis of a color map. Herein, the color map is a map that associates the size of elasticity data with hue information determined according to the three colors red (R), green (G), and blue (B). In this connection, red (R), green (G), and blue (B) have 256 tones, respectively, and as each color approaches the 255-th tone, the image is displayed with higher luminance. Further, as each color approaches the zero-th tone, the image is displayed with lower luminance.

For example, when displaying strain data, the color scan converter of the color DSC 115 converts into blue color code when the strain data output from the strain estimation processor 113 is small and converts into red color code when the strain data is large, and stores the data in the frame memory. When displaying elasticity data, the color scan converter of the color DSC 115 converts into blue color code when the elasticity data output from the elasticity estimation processor 114 is large and converts into red color code when the elasticity data is small, and stores the data in the frame memory. Subsequently, the image synthesizer 118 reads strain frame data or elasticity frame data with television synchronizing in accordance with a control command, and displays the resulting image on the display 119. Herein, in the elasticity image based on the strain frame data after the color tone conversion, a hard region (for example, a cancer, or an area with little strain) of the biological tissue is drawn with a blue color system, and a peripheral region of a soft region is drawn with a red color system. By viewing this type of elasticity image, for example, it is possible to visually grasp the spread and size of a cancer. The color DSC 115 can change the tints or the like of the color map in accordance with a command input via a user interface 117 such as a keyboard that is connected thereto via the controller 116.

The display system includes an image synthesizer 118 and a display 119 and the like. The image synthesizer 118 synthesizes a tomogram output from the tomography DSC 106 and an elasticity image output from the color DSC 115 to create one ultrasound image. For example, the image synthesizer 118 includes a frame memory, an image processor, and an image selecting part. Herein, the frame memory reads a tomogram output from the tomography DSC 106 and the elasticity image output from the color DSC 115, and, using a setting rate, adds and synthesizes luminance information and hue information for the pixels that mutually correspond on the same coordinate system of the tomogram and the elasticity image. More specifically, the image processor relatively superimposes the elasticity image on the tomogram using the same coordinate system. The image selecting part selects an image to be displayed on the display 119 from among a group of images stored in the frame memory in accordance with a control command. The display 119 has a monitor that displays the image data output from the image synthesizer 118.

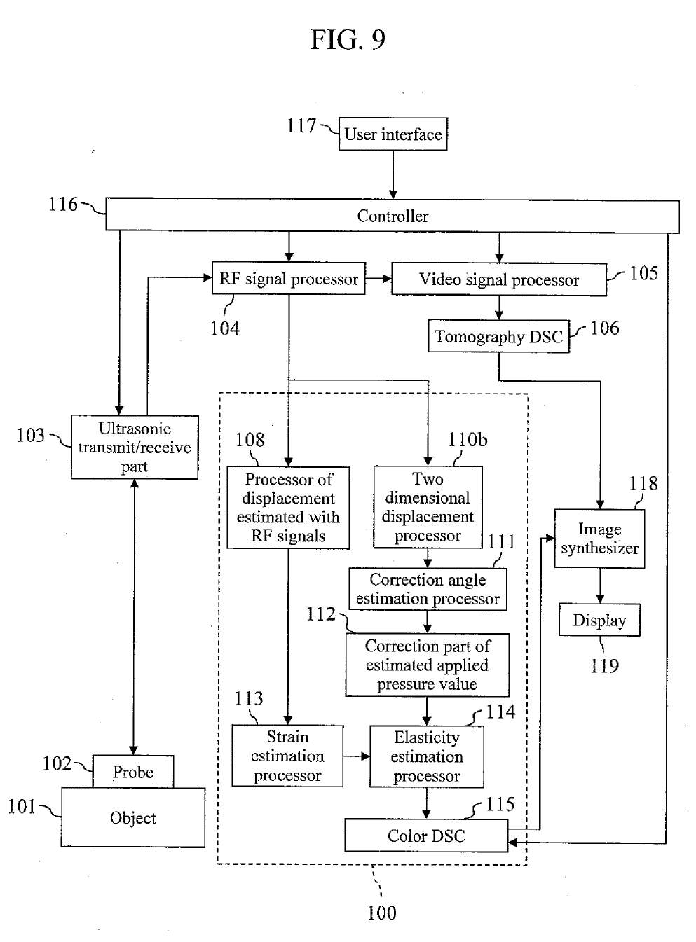

Although according to the present embodiment a two-dimensional displacement vector is determined based on a video signal, it is also possible to determine a displacement vector based on two-dimensional RF data using block matching or a cross-correlation function as shown in FIG. 9. Because the number of pieces of data increases, the estimation accuracy is improved. Normal video data is data obtained by performing wave detection from RF data and aligning a plurality of raster signals that have undergone log compression. In contrast, the term “two-dimensional RF data” refers to data in which a plurality of pieces of RF raster data are simply aligned, without performing wave detection or log compression or the like. Naturally, data for which some degree of resampling in the depth direction has been performed and for which the number of data points has been decreased is also included in the term “two-dimensional RF data” as used herein.

This is useful in a case where complex movements arise, such as when pressure is applied to the tissue of interest by an ultrasonic probe in a case in which, for example, an area in which the elasticity changes, such as bone, trachea, or intestinal tract is included at an inner part of the tissue of interest. Further, when there is a slipping surface (boundary surface of an organ or the like) between the pressure source and the measurement target region, since a force is applied via the slipping surface the movement direction is liable to be non-uniform. Although this kind of complicated movement does not constitute a significant problem at a mammary gland region or a prostate gland or the like, if strain imaging can also be performed with respect to the above described complicated movement, the objects for application thereof will expand. In that case, the risk that an error ascribable to the aforementioned deviation will be included in a measurement value will disappear. Hence, even when the uniformity of tissue displacement is poor, a modulus of elasticity image can be obtained with high accuracy.

### Embodiment 2

In Embodiment 1, a block matching method was used for a two-dimensional image in order to calculate a correction angle. In contrast, according to the present embodiment, a method is described that calculates a correction angle based on a bidirectional displacement measurement.

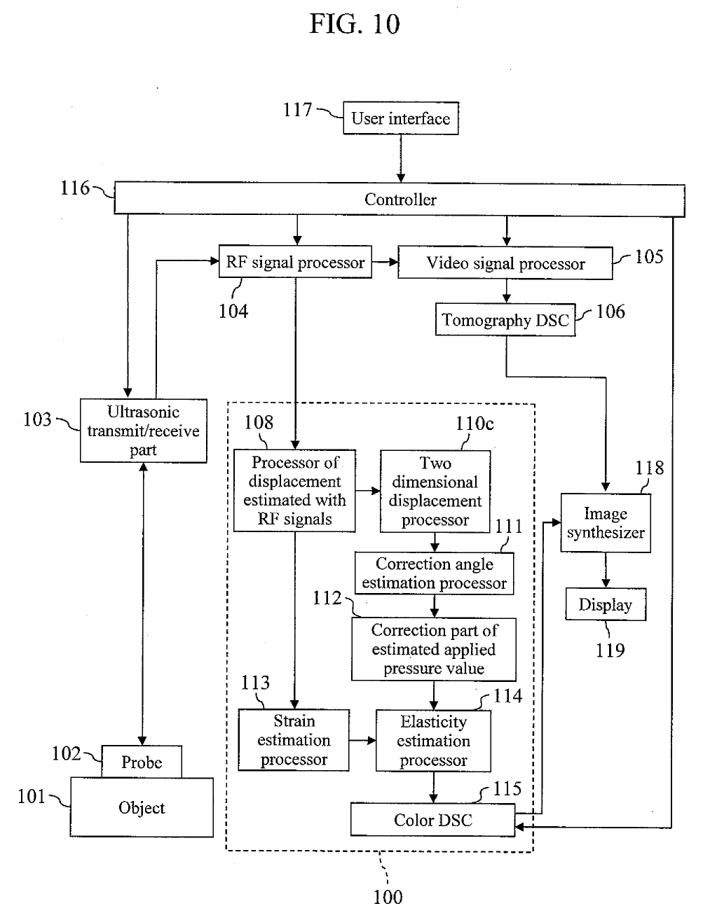

FIG. 10 is a block diagram of an ultrasonic diagnostic apparatus of the present embodiment. The apparatus illustrated in FIG. 10 differs from the apparatus configuration shown in FIG. 2 or FIG. 9 in the respect that a two dimensional displacement processor 110c estimates a two-dimensional displacement based on data output from the processor of displacement estimated with RF signals 108.

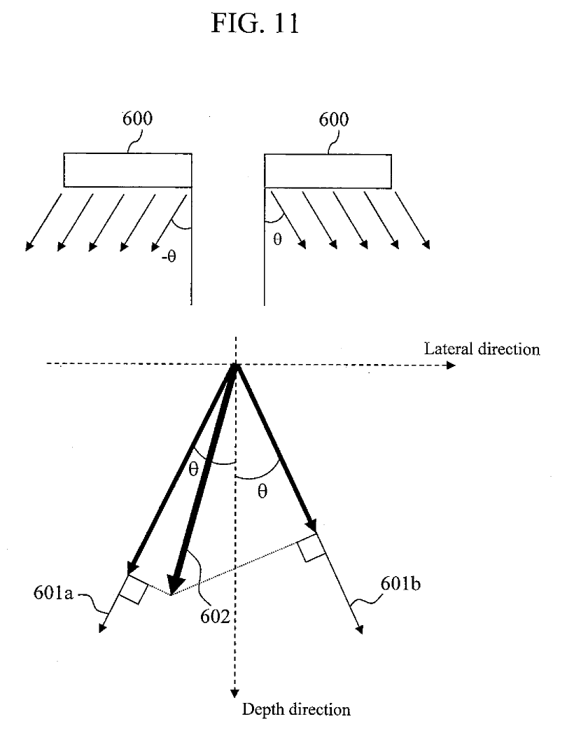

First, two-way ultrasonic transmission is described using FIG. 11. In ultrasonic imaging performed by electronic scanning using a phased array 600, by controlling a delay time between elements it is possible to perform transmission and reception of waves in which an ultrasound beam is not transmitted not only from the front face but is also deflected by an angle θ from the front face. Therefore, a direction that forms an angle −θ with a direction that is perpendicular to a surface that faces the object of an element is regarded as a first measurement direction (measurement vector 1 direction) 601a. Imaging of frame 1 is performed a plurality of times (for example, two times) before and after deformation of the object with this steering, and a displacement in the angle −θ direction is determined using the correlation between the frames (displacement in displacement search vector 1 direction). Next, imagining in a second measurement direction (measurement vector 2 direction) 601b is performed before and after deformation of the object, and a displacement in the angle +θ direction is determined using the correlation between the frames (displacement in displacement search vector 2 direction). A displacement two-dimensional vector corresponding to the two-dimensional strain is determined by addition processing with respect to the measurement vector 1 and measurement vector 2.

If the measurement vector 1 and the measurement vector 2 are orthogonal, it is easy to obtain a displacement two-dimensional vector by adding the two measurement vectors. However, in ultrasonic imaging, if the steering angle is too large there is the possibility of increasing artifacts caused by a grating beam. Therefore, the steering angle may be set to less than 45 degrees, and preferably from 20 to 30 degrees. Further, additional lines may be drawn in an orthogonal direction to the two measurement vectors, respectively, and a displacement two-dimensional vector 602 may be determined that takes an intersection point thereof as an end point.

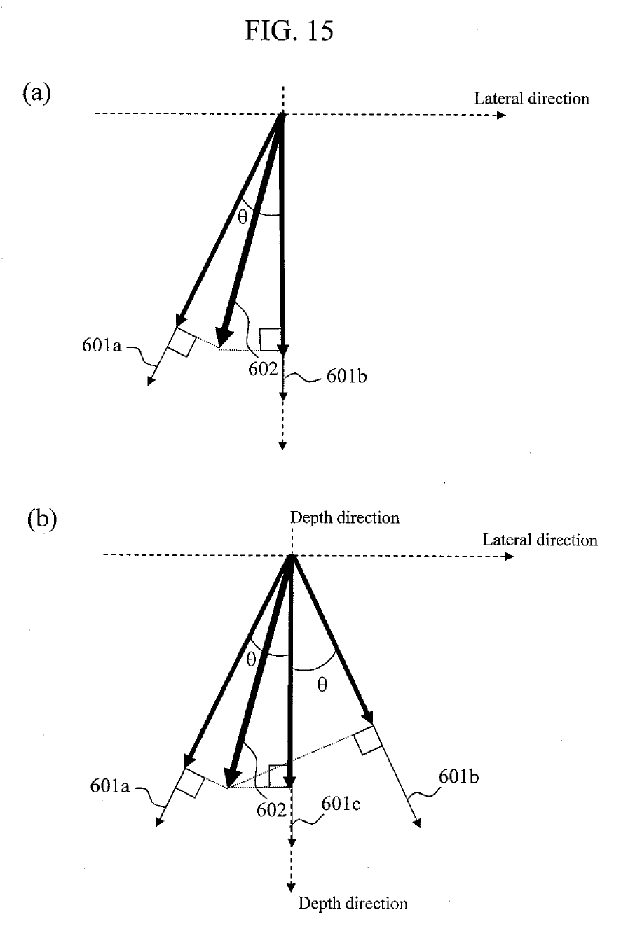

Although an example of steering angles that open at an angle θ to the left and right, respectively, of the center of a normal line vector of a wave transmitting surface is described according to FIG. 11, a configuration may also be adopted, as shown in FIG. 15(a), such that left-right asymmetry is set as in the case of a combination of a normal line vector and a steering angle of angle θ. When the steering angle is 0 degrees, since the grating angle is minimized, the acoustic signal-noise ratio can be made as small as possible. Although according to FIG. 15(a) a combination of the normal line vector and a steering angle to the left is used, from the viewpoint of symmetry, the combinations may be swung to the left and right alternately. Further, as shown in FIG. 15(b), three or more steering angles may be used for measuring the moving direction. In this case, although the frame rate decreases, the estimation accuracy with respect to the displacement detection can be enhanced. Although in this case there may be two or more intersection point positions, in such case a single intersection point can be determined by using the mean value thereof.

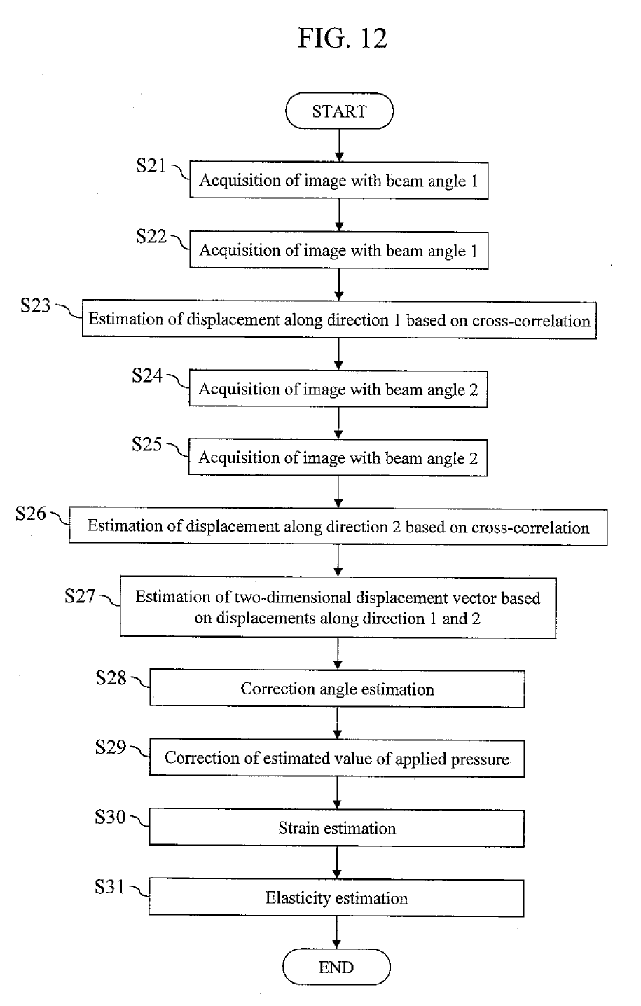

FIG. 12 is a flowchart of processing according to the present embodiment. RF data is acquired before and after deformation of the object with respect to the first measurement direction 601a (S21, S22), and a displacement along the first measurement direction 601a is estimated by a cross-correlation operation (S23). Next, RF data is acquired before and after deformation of the object with respect to the second measurement direction 601b (S24, S25), and a displacement along the first measurement direction 601a is similarly estimated by a cross-correlation operation (S26). Next, a two-dimensional displacement vector is estimated based on the displacements along the first measurement direction 601a and along the second measurement direction 601b (S27). After determining a two-dimensional displacement vector based on an RF correlation relating to two different directions in this manner, the method described in Embodiment 1 is used to perform angle correction (S28), correction of the estimated applied pressure value (S29), and estimate the elasticity based on a strain estimation result that is previously determined (determined based on the image in the direction in which θ is 0 in FIG. 11 (S30, S31). According to the present method, although the frame rate decreases compared to Embodiment 1, the displacement vector estimation accuracy can be improved.

Although embodiments of an ultrasonic diagnostic apparatus to which the present invention is applied have been described in the foregoing, an ultrasonic diagnostic apparatus that applies the present invention can be embodied in various different forms without departing from the technical spirit or essential features of the present invention. Therefore, it should be understood that the foregoing embodiments are merely illustrative in all aspects and are not to be construed as limiting the present invention.

1. An ultrasonic imaging system, comprising:
an ultrasonic probe that transmits an ultrasonic wave at an object and receives a reflection echo;
an RF signal processor that acquires a first RF raster signal corresponding to an ultrasonic wave that is transmitted before a deformation of interest of an object, and a second RF raster signal corresponding to an ultrasonic wave that is transmitted after the deformation of interest;
a processor of displacement estimated with RF signals that acquires a displacement in a raster direction of each portion of an object based on the first RF raster signal and second RF raster signal;
a two dimensional displacement processor that estimates a two-dimensional displacement vector that shows a displacement of each portion before and after the deformation of interest;
an applied pressure estimated value correction part that corrects an applied pressure estimated value produced by the deformation of interest in correspondence with a direction of the estimated two-dimensional displacement vector and an ultrasonic wave irradiation direction;
a strain estimation part that estimates a strain of each portion of the object based on the corrected applied pressure estimated value and the two-dimensional displacement vector; and
a display that displays strain information that is estimated by the strain estimation part.

2. The ultrasonic imaging system according to claim 1, wherein the applied pressure estimated value correction part determines an angle formed by the two-dimensional displacement vector and an ultrasonic wave irradiation direction at each portion of the object as a correction angle, and corrects an applied pressure estimated value of the displacement of interest based on the correction angle that is determined.

3. The ultrasonic imaging system according to claim 2, further comprising:
an elasticity estimation processor that spatially differentiates the displacement to estimate elasticity information of each portion of an object;
wherein the elasticity estimation processor estimates an elasticity of each portion of an object based on the corrected applied pressure estimated value and the strain information.

4. The ultrasonic imaging system according to claim 1, wherein the two dimensional displacement processor divides an ultrasound image frame of the object before the deformation of interest and an ultrasound image frame of the object after the deformation of interest into a plurality of areas, respectively, and estimates a two-dimensional displacement vector of each area by comparing the areas of the two frames.

5. The ultrasonic imaging system according to claim 1, wherein the two dimensional displacement processor estimates a two-dimensional displacement vector based on a first displacement that is determined by means of ultrasonic waves that are transmitted and received along a first direction, and a second displacement that is determined by means of ultrasonic waves that are transmitted and received along a second direction that is different from the first direction.

6. The ultrasonic imaging system according to claim 1, wherein the two dimensional displacement processor also determines a displacement vector by means of block matching or a cross-correlation function based on two-dimensional RF data.

7. The ultrasonic imaging system according to claim 1, wherein the RF signal processor includes a memory that stores RF signal data in time series that is received by means of the ultrasonic probe, and an RF signal selection part that selects a set of the stored RF raster signals in adjoining frames on two time axes.

8. The ultrasonic imaging system according to claim 7, wherein the RF signal selection part sets a cross correlation window that limits a depth in an RF raster signal of a first frame, and sets a searching area that limits a depth in an RF raster signal of a second frame.

9. The ultrasonic imaging system according to claim 2, wherein the correction angle is set to less than 45 degrees, and preferably to from 20 degrees to 30 degrees.

10. The ultrasonic imaging system according to claim 9, wherein the displacement two-dimensional vector is determined based on a vector that takes an intersection point between additional lines that are drawn in an orthogonal direction to the two measurement vectors, respectively, as an end point.

11. The ultrasonic imaging system according to claim 5, wherein, one of the first direction and the second direction matches a normal line direction of a wave transmitting surface of the ultrasonic probe.

12. The ultrasonic imaging system according to claim 11, wherein, a direction among the first direction and the second direction that does not match a normal line direction of the wave transmitting surface of the ultrasonic probe is set so as to be different for each frame.

13. The ultrasonic imaging system according to claim 1, wherein the two dimensional displacement processor performs transmitting and receiving of ultrasonic waves with respect to three or more directions, and estimates a single two-dimensional displacement vector based on displacements in each direction that are determined.
